(19) United States
(12) Patent Application Publication
(10) Pub. No.: US 2003/0198692 A1
(43) Pub. Date: Oct. 23, 2003

(54) Microbial inhibitory compositions

(76) Inventors: Gerd Pia Carola Holmstrom, Randwick (AU); Staffan Kjellberg, La Perouse (AU)

Correspondence Address: NIXON & VANDERHYE, PC, 1100 N GLEBE ROAD, 8TH FLOOR, ARLINGTON, VA, 22201-4714, US

(21) Appl. No.: 10/221,675

(22) PCT Filed: Mar. 16, 2001
(86) PCT No.: PCT/AU01/00295

(30) Foreign Application Priority Data
Mar. 16, 2000 (AU) PQ 6292

(51) Int. Cl. A61K 31/365; A61K 31/44; A61K 31/19; A61K 33/40; A01N 33/12; A61K 33/36; A61K 33/14

(52) U.S. Cl. 424/661; 514/473; 424/616; 424/669; 514/557; 514/358; 514/642

(57) ABSTRACT

The present invention provides an antimicrobial composition. The composition comprises a cell-permeabilising agent and at least one compound of general formula (I) wherein R₁ and R₂ are independently H, halogen, alkyl, alkoxy, oxoalkyl, alkenyl, aryl or arylalkyl whether unsubstituted or substituted, optionally interrupted by one or more heteroatoms, straight chain or branched chain, hydrophilic fluorophilic; R₃ and R₄ are independently H, halogen, alkyl, aryl or arylalkyl, alkoxy; R₃ or R₄+R₂ can be saturated or an unsaturated cycloalkane; and “ [symbol] ” represents a single bond or a double bond provided that at least one of R₁, R₂, R₃ and R₄ is a halogen.

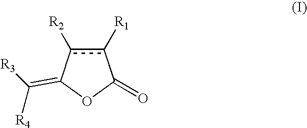

### FIELD OF THE INVENTION

The present invention relates to compositions for use in inhibiting microorganisms.

### BACKGROUND OF THE INVENTION

It is known that a variety of furanone compounds possessing antifungal and antimicrobial properties can be isolated from red marine algae Delisea fimbriata, Delisea alegans and Delisea pulchra (Reichelt and Borowitzka (1984) Hydrobiologia 116; 158-168). When first isolated, it was thought that these compounds may be suitable as antimicrobial agents for use in animals including humans. Unfortunately, it was found that most if not all of these naturally occurring compounds were toxic to animal cells at the concentrations required to inhibit microorganisms and therefore unsuitable for many veterinary and medical applications.

Gram positive bacteria are a major problem in hospitals, on skin, in the dental area, for heart transplants, catheters, and other biomedical implants. Unfortunately, not all antimicrobial agents are active against Gram positive bacteria. Gram positive bacteria are also present in domestic areas including bathrooms, toilets and kitchens and can also cause a disease hazard for these sources. Accordingly, there is a need for more agents that are suitable to inhibit or kill these types of microorganisms in many varied situations including domestic, veterinary and medical applications.

Gram negative bacteria also pose a threat to human and animal health and new agents are also required to inhibit these microorganisms.

Fungi are a major problem in hospitals, on skin, in the dental area, for heart transplants, catheters, and other biomedical implants. Fungi are also present in domestic areas including bathrooms, toilets and kitchens and can also cause a disease hazard for these sources. Unfortunately, only a few antifungal agents are available which have broad spectrum of activity. Accordingly, there is a need for more agents that are suitable to inhibit or kill fungi in many varied situations including domestic, veterinary and medical applications.

The present inventors have now made the surprising finding that active antimicrobial compositions which inhibit microbial growth can be prepared using a mixture of one or several furanone compounds, many of which were previously believed not to be suitable as antimicrobial agents.

### SUMMARY OF THE INVENTION

In a first aspect, the present invention consists in an antimicrobial composition, the composition comprising a cell-permeabilising agent and at least one compound of general formula I:

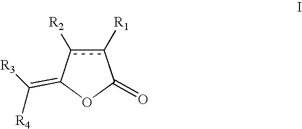

wherein R₁ and R₂ are independently H, halogen, alkyl, alkoxy, oxoalkyl, alkenyl, aryl or arylalkyl whether unsubstituted or substituted, optionally interrupted by one or more heteroatoms, straight chain or branched chain, hydrophilic or fluorophilic;

R₃ and R₄ are independently H, halogen, alkyl, aryl or arylalkyl, alkoxy;

R₃ or R₄+R₂ can be a saturated or an unsaturated cycloalkane;

and “ [symbol] ” represents a single bond or a double bond provided that at least one of R₁, R₂, R₃ and R₄ is halogen.

Preferably, at least one of R₁, R₂, R₃ and R₄ is bromine. Most preferably, at least one of R₃ and R₄ is Br.

The term “alkyl” is taken to mean both straight chain alkyl groups such as methyl, ethyl, propyl, isopropyl, butyl, isobutyl, sec-butyl, tertiary butyl, and the like. Preferably the alkyl group is a lower alkyl of 1 to 6 carbon atoms. The alkyl group may optionally be substituted by one or more groups selected from alkyl, cycloalkyl, alkenyl, alkynyl, halo, haloalkyl, haloalkynyl, hydroxy, alkoxy, alkenyloxy, haloalkoxy, haloalkenyloxy, nitro, amino, nitroalkyl, nitroalkenyl, nitroalkynyl, nitroheterocyclyl, alkylamino, dialkylamino, alkenylamine, alkynylamino, acyl, alkenoyl, alkynoyl, acylamino, diacylamino, acyloxy, alkylsulfonyloxy, heterocyclyl, heterocycloxy, heterocyclamino, haloheterocyclyl, alkylsulfenyl, alkylcarbonyloxy, alkylthio, acylthio, phosphorus-containing groups such as phosphono and phosphinyl. The alkyl group may also be perflourinated.

The term “alkoxy” denotes straight chain or branched alkyloxy, preferably C₁₋₁₀ alkoxy. Examples include methoxy, ethoxy, n-propoxy, isopropoxy and the different butoxy isomers.

The term “alkenyl” denotes groups formed from straight chain, branched or mono- or polycyclic alkenes and polyene. Substituents include mono- or poly-unsaturated alkyl or cycloalkyl groups as previously defined, preferably C₂₋₁₀ alkenyl. Examples of alkenyl include vinyl, ally, 1-methylvinyl, butenyl, iso-butenyl, 3-methyl-2-butenyl, 1-pentenyl, cyclopentenyl, 1-methyl-cyclopentenyl, 1-hexenyl, 3-hexenyl, cyclohexenyl, 1-heptenyl, 3-heptenyl, 1-octenyl, cyclooctenyl, 1-nonenyl, 2-nonenyl, 3-nonenyl, 1-decenyl, 3-decenyl, 1,3-butadienyl, 1-4,pentadienyl, 1,3-cyclopentadienyl, 1,3-hexadienyl, 1,4-hexadienyl, 1,3-cyclohexadienyl, 1,4-cyclohexadienyl, 1,3-cycloheptadienyl, 1,3,5-cycloheptatrienyl, or 1,3,5,7-cyclooctatetraenyl.

The term “halogen” denotes fluorine, chlorine, bromine or iodine, preferably bromine or fluorine.

The term “heteroatoms” denotes O, N or S.

The term “acyl” used either alone or in compound words such as “acyloxy”, “acylthio”, “acylamino” or diacylamino” denotes an aliphatic acyl group and an acyl group containing a heterocyclic ring which is referred to as heterocyclic acyl, preferably a C₁₋₁₀ alkanoyl. Examples of acyl include carbamoyl; straight chain or branched alkanoyl, such as formyl, acetyl, propanoyl, butanoyl, 2-methylpropanoyl, pentanoyl, 2,2-dimethylpropanoyl, hexanoyl, heptanoyl, octanoyl, nonanoyl, decanoyl; alkoxycarbonyl, such as methoxycarbonyl, ethoxycarbonyl, t-butoxycarbonyl, t-pentyloxycarbonyl or heptyloxycarbonyl; cycloalkanecarbonyl such as cyclopropanecarbonyl cyclobutanecarbonyl, cyclopentanecarbonyl or cyclohexanecarbonyl; alkanesulfonyl, such as methanesulfonyl or ethanesulfonyl; alkoxysulfonyl, such as methoxysulfonyl or ethoxysulfonyl; heterocycloalkanecarbonyl; heterocyclyoalkanoyl, such as pyrrolidinylacetyl, pyrrolidinylpropanoyl, pyrrolidinylbutanoyl, pyrrolidinylpentanoyl, pyrrolidinylhexanoyl or thiazolidinylacetyl; heterocyclylalkenoyl, such as heterocyclylpropenoyl, heterocyclylbutenoyl, heterocyclylpentenoyl or heterocyclylhexenoyl; or heterocyclylglyoxyloyl, such as, thiazolidinylglyoxyloyl or pyrrolidinylglyoxyloyl.

As will be recognised by those skilled in the art the compounds of general formula I can exist as two isomers E and Z. It is intended that the general formulas depicted herein are not limited to a particular isomer and encompass both isomers either in the form of a racemic mixture or separated stereo isomers.

As used herein the term “cell-permeabilising agent” is used in its broadest sense and means an agent which increases the permeability of the cell membrane and/or cell wall of bacteria, yeast and fungi. A number of such agents are well known in the field and include certain antibiotics, aldehydes, biguanides, halogen releasing agents, peroxygens, phenols, bis-phenols, quaternary ammonium compounds, alcohols, glycols, ionic and non-ionic detergents.

Examples of suitable cell-permeabilising agents for combination with furanone compounds according to the present invention are set out in Table 1.

TABLE 1
Cell-permeabilising agents

| Class of agent | Examples |
|---|---|
| Antibiotic | Polymyxin B |
| Aldehydes | Glutaraldehyde |
| | Formaldehyde |
| Biguanides | Chlorhexidine |
| Halogen releasing agents | Hypochlorous acid |
| | Iodine |
| Peroxygens | Hydrogen peroxide |
| Phenols | Chlorhexidine |
| | Peracetic acid |
| Bis-Phenols | Chlorinated bis-phenol fenticlor |
| | Hexachlorophene |
| Quaternary ammonium | Cetyltrimethylammonium bromide |
| compounds | (CTAB) |
| | Tetrabutlammoniumhydrogen sulfate |
| | Didecyldimethylammonium bromide |
| | Cetylpyridium chloride |
| Alcohols | Toluene |
| Glycols | Polyethylene glycol (PEG) |
| Ionic detergent | Ethylenediaminetetraacetic acid |
| | (EDTA) |
| | Diamidines |
| | Citric acid |
| | Sodium lauryl sulfate (SDS) |
| Non-ionic detergent | TritonX-100 |
| | Tween 80 |

In a preferred embodiment of the present invention compound is selected from the group consisting of

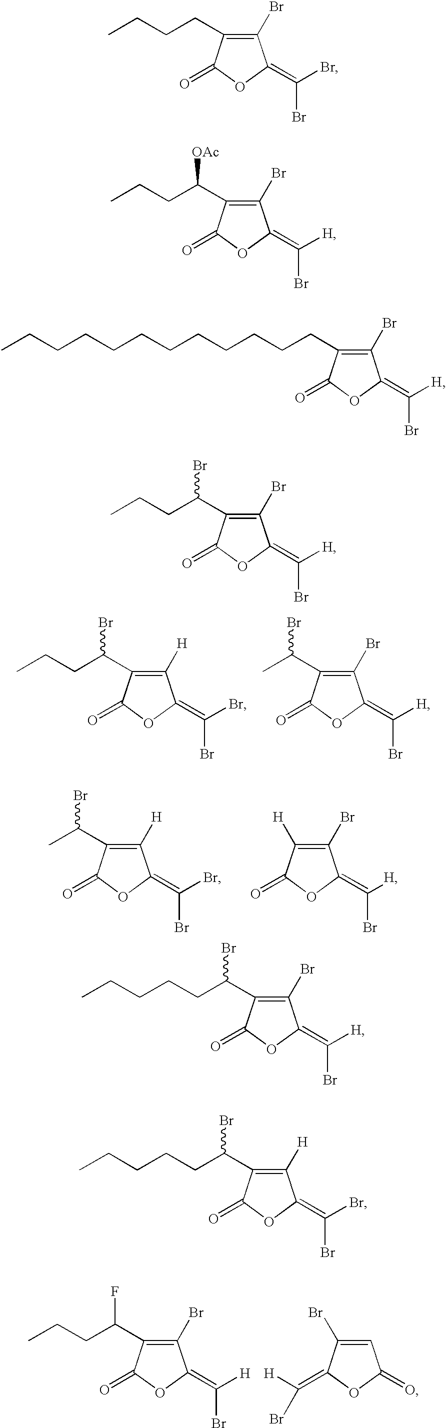

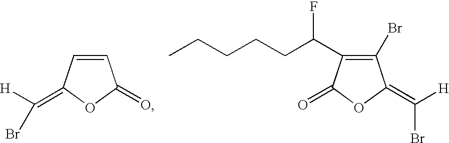

and combinations thereof.

For ease of reference these compounds will be referred to hereafter as compounds 2, 3, 19, 24, 25, 26, 27, 30, 33, 34, 45, 55, 56 and 57 as set out in Table 2.

TABLE 2

| Compound | Structure |
|---|---|
| 2 | 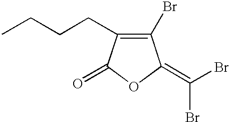 |
| 3 | 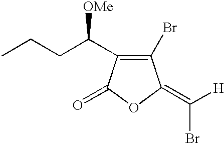 |
| 19 | 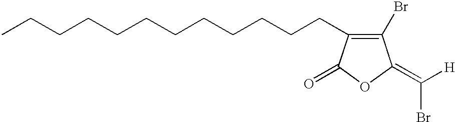 |
| 24 | 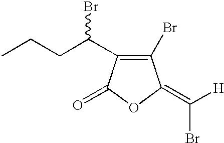 |
| 25 | 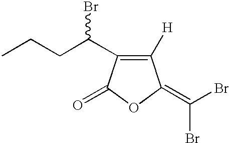 |
| 26 | 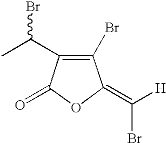 |
| 27 | 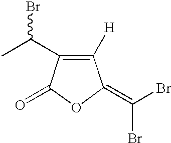 |
| 30 | 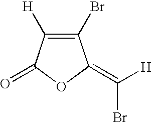 |
| 33 | 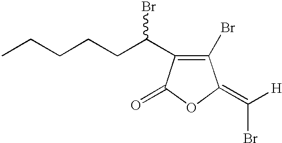 |
| 34 | 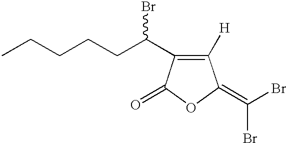 |
| 45 | 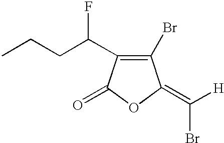 |
| 55 | 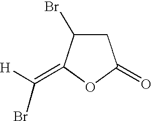 |
| 56 | 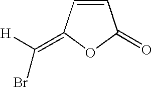 |
| 57 | 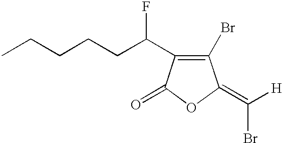 |

The concentration of the compound or mixture of compounds in the composition is preferably between about 100 ng/ml and 100 μg/ml.

In use, the concentration of the furanone compound or mixture of furanone compounds in the presence of the cell-permeabilising agent required to have activity against bacteria is typically about 10 μg/ml.

The composition can be active against bacteria, yeasts and fungi.

Preferably, the cell-permeabilising agent is selected from antibiotics, chelating agents, ionic detergents, non-ionic detergents, organic solvents, quaternary ammonium compounds, and glycols.

Preferably, the antibiotic is polymyxin B, the chelating agent is N,N′-1,2-ethanediylbis[N-(carboxy-methyl)glycine] (EDTA), the ionic detergent is sodium lauryl sulfate (SDS) or cetyltrimethylammonium bromide (CTAB), the non-ionic detergent is TritonX-100 or Tween 80, the organic solvent is toluene, quaternary ammonium compound is cetylpyridinium chloride, and the glycol is polyethylene glycol (PEG).

The concentration of the cell-permeabilising agent can vary, depending on the agent used. For example, it has been found by the present inventors that 0.5 μg/ml of polymyxin B is particularly suitable. Similarly, 0.02% EDTA was found to be effective in a number of compositions.

In a second aspect, the present invention consists in a method of manufacturing an antimicrobial composition, the method comprising combining a compound of general formula I or a mixture of two or more such compounds with a cell-permeabilising agent and a pharmaceutically acceptable diluent.

In a third aspect, the present invention consists in a method of inhibiting the growth of a microorganism, the method comprising exposing the microorganism to an effective amount of an antimicrobial composition according to the first aspect of the present invention for sufficient time such that the microorganism is inhibited.

In a fourth aspect the present invention consists in a method of treating bacterial infection or decreasing the severity of symptoms of bacterial infection in an animal, the method comprising administering to the animal an effective amount of the composition of the first aspect of the present invention.

The method includes in vivo and in vitro treatment of microorganisms. The composition may be formulated as a pharmaceutical agent for human and animal use, a topical agent for human and animal use, a disinfectant, an antiseptic, a mouth wash or rinse, a soap or cleaning agent or as part of animal feedstocks. The general formulations used for such products, in particular disinfectants, antiseptics, dentifrices, mouth washes or rinses, soaps, cleaning agents and supplements for animal feedstocks, is well known in the art. The compositions of the present invention can be advantageously incorporated in such formulations, or alternatively the compositions of the present invention can further comprise ingredients which make up such products.

The composition of the present invention may also be used in the cleaning a surface, such as a hard surface, woven surface or non-woven surface. Examples of surfaces in the cleaning of the composition of the present invention may be advantageously employed include toilet bowls, bath tubs, drains, countertops, food surfaces, airducts, air conditioners, carpets or cloths. The composition of the present invention may also be used in paints so as to provide a microbial inhibitory property to the paint.

The compositions according to the present invention are particularly suitable for use in the treatment of cystic fibrosis, Pseudomonas infections, Candida infections, persistent burns infections, wound infections, contact lens cleaning solutions, skin creams, treatment of oral infections, fungicides and a variety of other inhibitory products. It will be appreciated that the compositions can be used or may be applicable in any situation where microbial inhibition is required.

The composition of the present invention can also be formulated in a topical dressing for burns.

The composition of the present invention can be used in environmental, sanitary, veterinary, or medical applications to inhibit the growth of microbes.

Applications include, but are not limited to, inhibition of growth of microbial pathogens in environmental situations, reduction or prevention of microbial colonisation of medical media including washing solutions, ointments and the like, inhibition of microbial attachment to surfaces and subsequent biofilm formation, as active ingredients in antiseptics and disinfectants.

The compositions of the present invention will also find application in preventing or inhibiting biofilm formation. In another embodiment the compositions will find application as washing solutions, particularly in contact lens cleaning compositions.

The ability of composition of the present invention to inhibit the growth of a range of microbes provides a number of useful applications of these compositions. In particular the compositions may be formulated for pharmaceutical use with human and non-human animals. In one embodiment of the invention the compositions are formulated for topical application for use, for example, in application to wounds and the like. In this regard they may be directly incorporated into bandages and the like.

In a further aspect the present invention consists in a method of treating Pseudomonas infection in an animal, the method comprising administering to an animal in need of such treatment a composition comprising tobramycin and at least one compound of general formula I as defined above.

In a preferred embodiment the Pseudomonas infection is a lung infection, in particular P. aeruginosa infection. In this embodiment it is preferred that the composition is administered by inhalation.

In a furthered preferred embodiment it is preferred that the animal is human. In one embodiment the animal is suffering from cystic fibrosis.

In a still further aspect the present invention consists in a composition for use in treatment of Pseudomonas infection, the composition comprising tobramycin and at least one compound of general formula I as defined above.

Throughout this specification, unless the context requires otherwise, the word “comprise”, or variations such as “comprises” or “comprising”, will be understood to imply the inclusion of a stated element, integer or step, or group of elements, integers or steps, but not the exclusion of any other element, integer or step, or group of elements, integers or steps.

### DETAILED DESCRIPTION

In order that the present invention may be more clearly understood, preferred forms will be described with reference to the following examples and drawings.

### BRIEF DESCRIPTION OF DRAWINGS

FIG. 1. Growth of Pseudomonas aeruginosa in the presence of various furanones.

FIG. 2. Screening of different furanones in the presence of polymyxin with Escherichia coli.

FIG. 3. Growth of Burkholdera cepacia in the presence of polymyxin B and various furanones.

FIG. 4. Growth of Pseudomonas aeruginosa against polymyxin B and various furanones.

FIG. 5. Growth of Pseudomonas aeruginosa in the presence of EDTA and various furanones.

FIG. 6. Growth of Pseudomonas aeruginosa in the presence of citric acid and furanone 30.

FIG. 7. Growth of Pseudomonas aeruginosa in the presence of citric acid and furanone 34.

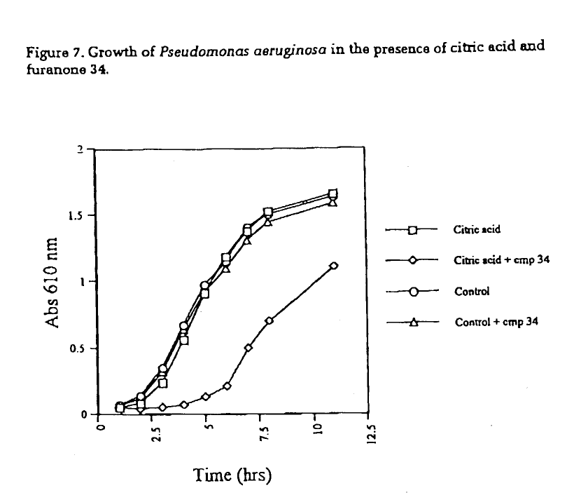

FIG. 8. Growth of Pseudomonas aeruginosa in the presence of tetrabutylammoniumhydrogen sulfate and furanone 30.

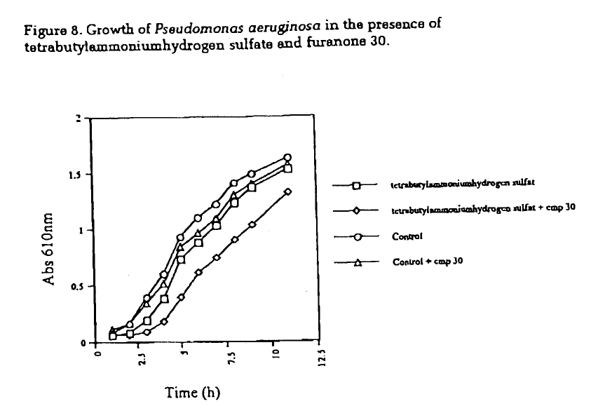

FIG. 9. Growth of Pseudomonas aeruginosa in the presence of didecldimethylammonium bromide and furanone 30.

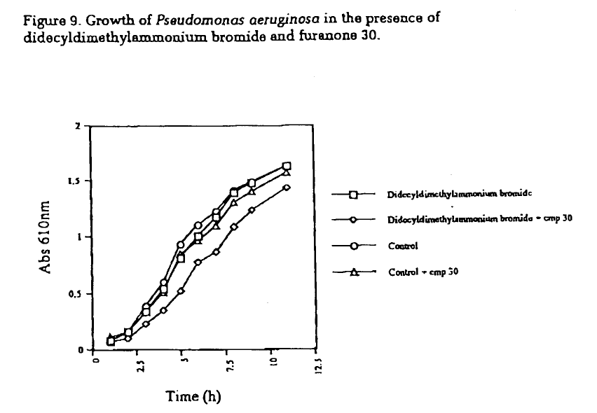

FIG. 10. Growth of Pseudomonas aeruginosa in the presence of Tween 80 and compound 30.

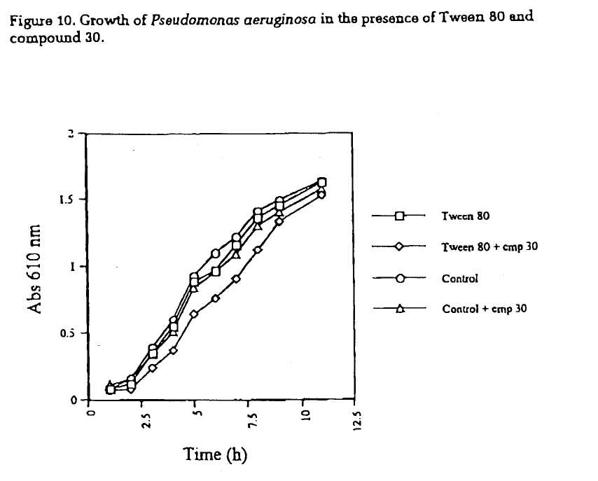

FIG. 11. Growth of Corynebacterium jeikeium in the presence of furanone 2 and EDTA.

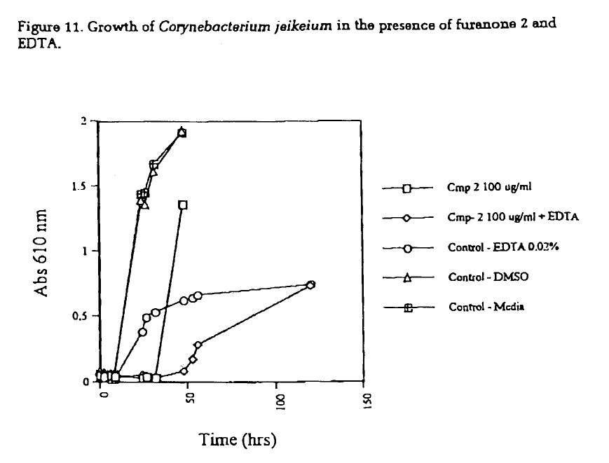

FIG. 12. Growth of Candida albicans in the presence of EDTA and furanone 57.

### EXAMPLE 1

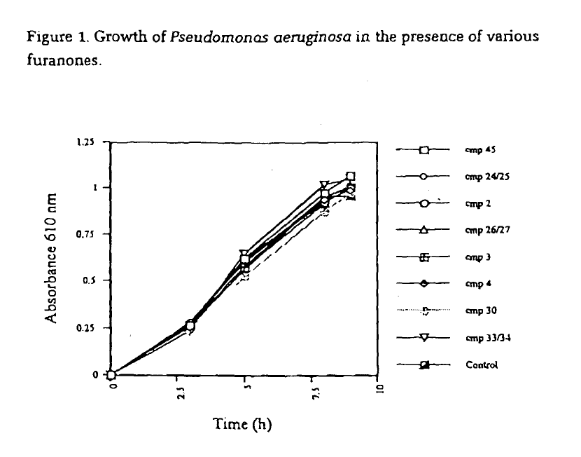

Ten furanones (compounds 2, 3, 19, 30, 45, 55, 56, 24/25, 26/27 and 33/34) (see Table 2) were tested against growth of Gram negative bacteria in a combination treatment using a cell permeabilising agent (Polymyxin B and EDTA). As can be seen in FIG. 1 the growth of Pseudomonas aeruginosa was not affected by the different furanones alone.

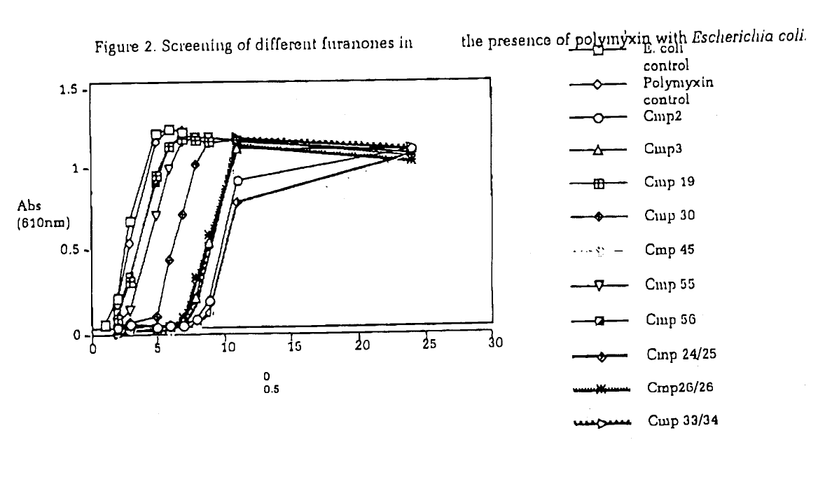

Growth of Gram negative bacteria is not generally affected by furanone compounds alone. However, by simultaneously adding a compound which interferes with the permeability of the cell membrane, the present inventors have found that furanone compounds in combination with a permeability agent can prevent growth of microorganisms including bacteria, particularly Gram negative bacteria. In order to explore this concept, the antibiotic polymyxin B was included in the initial round of experiments (see FIGS. 2, 3 and 4) involving the bacteria Escherichia coli, Burkholdera cepacia and Pseudomonas aeruginosa. The results from these experiments suggested that different furanone compounds target different Gram negative bacterial strains.

Different furanone compounds under test were applied at 10 μg/ml (concentration of stock solution of furanone compound or mixture of compounds was 2 mg/ml) and polymyxin B was employed at concentrations which ranged from 0.3-1 μg/ml (stock solution was 10 mg/ml).

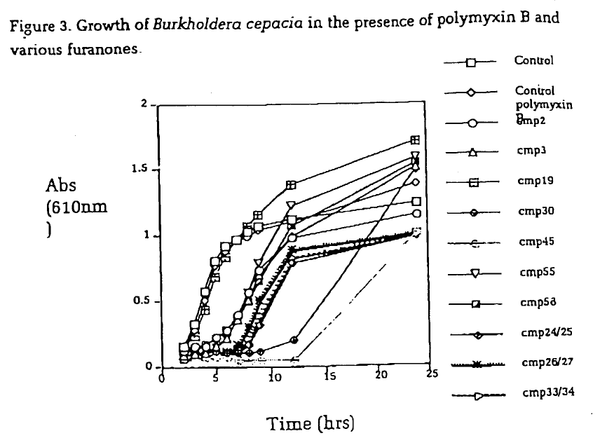

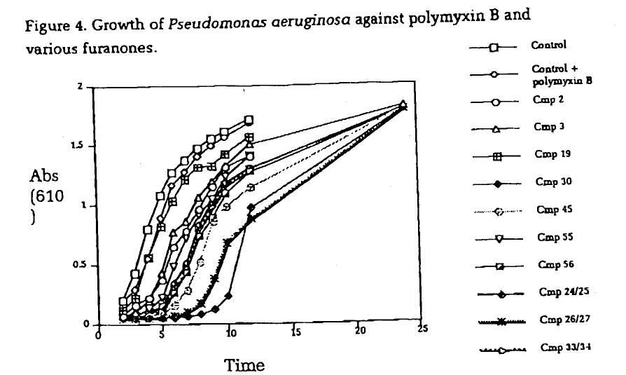

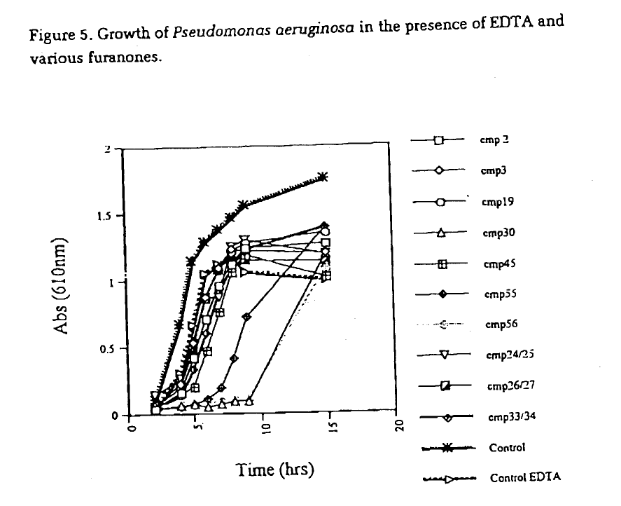

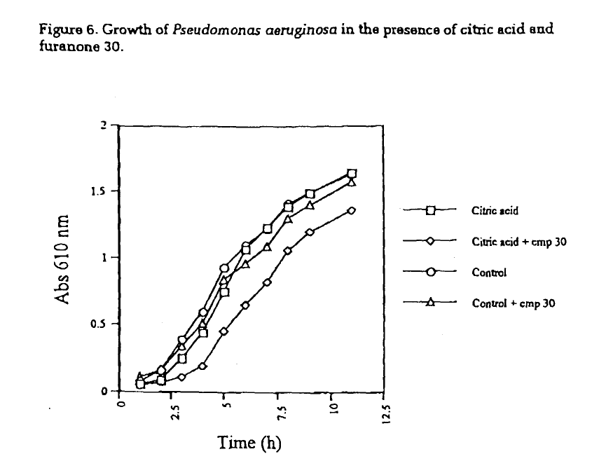

The results showed that compounds 45, 24/25 and 2 inhibited the growth of E. coli for a time period of 8 hr (FIG. 3) and compounds 45 and 30 prolonged the lag phase of growth of B. cepacia for 8-10 hr (FIG. 4). Compound 30 was demonstrated to be the most active compound against P. aeruginosa (FIG. 5). In addition, EDTA which also affects the permeability of the cell membrane was tested against growth of P. aeruginosa in combination with the different furanones. EDTA was added at a concentration of 0.02%. The results demonstrated that compounds 30 and 56 were the most effective compounds in preventing growth (FIG. 6) for this organism. These results suggest that the mode of actions of polymyxin B and EDTA are different. It is possible that they differently allow for different furanones to penetrate the cell membrane.

### EXAMPLE 2

Growth of Pseudomonas aeruginosa in the presence of citric acid, toluene, Tween 80 and two different quaternary ammonium compounds was further investigated. The tested furanones was furanone 30 and 34 at 10 μg/ml (concentration of stock solution of furanone compound was 10 mg/ml). The used cell-permeability agents were employed at concentrations which ranged from 0.35-0.001%.

The results demonstrated that compound 34 in combination with citric acid prolonged the lag phase of growth with approximately 3 hours. Compound 30+citric acid also prolonged the lag phase of growth however not as strongly as compound 34. These results support the data from FIGS. 2-5 that different furanones in combination with a cell-permeability agent act differently on the growth of microorganisms. The two tested quaternary ammonium compounds in combination with furanone 30 inhibited the growth of P. aeruginosa with tetrabutylammoniumhydrogen sulfate being slightly more active compared to didecyldimethylammonium bromide. The cell-permeability agent, Tween 80, gave a slight growth inhibition in combination with compound 30. Moreover, the growth of the Gram-positive bacteria, Corynebacterium jeikeium, was inhibited by compound 2 (100 μg/ml) in combination with EDTA (0.02%). The lagphase of growth was prolonged for 20 hr.

### EXAMPLE 3

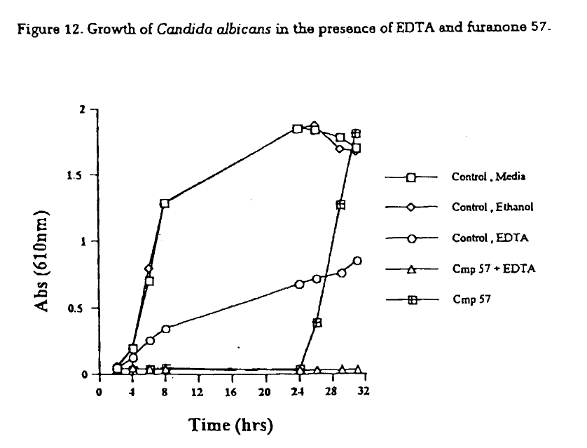

The yeast, Candida albicans, was tested in the presence of furanone 57 at 250 ng/ml. The used cell-permeability agent was EDTA (0.01%) and the results are shown in FIG. 12. The result demonstrated that compound 57 (250 ng/ml) inhibited the growth of C. albicans cells for 24 hrs. In combination with 0.01% EDTA the growth of the cells was inhibited for at least 32 hrs.

It will be appreciated by persons skilled in the art that numerous variations and/or modifications may be made to the invention as shown in the specific embodiments without departing from the spirit or scope of the invention as broadly described. The present embodiments are, therefore, to be considered in all respects as illustrative and not restrictive.

1. An antimicrobial composition, the composition comprising a cell-permeabilising agent and at least one compound of general formula I: 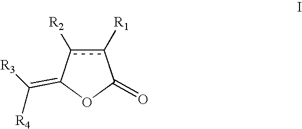
wherein R₁ and R₂ are independently H, halogen, alky, alkoxy, oxoalkyl, alkenyl, aryl or arylalkyl whether unsubstituted or substituted, optionally interrupted by one or more heteroatoms, straight chain or branched chain, hydrophilic or fluorophilic;
R₃ and R₄ are independently H, halogen, alkyl, aryl or arylalkyl, alkoxy;
R₃ or R₄+R₂ can be a saturated or an unsaturated cycloalkane;
and “ [symbol] ” represents a single bond or a double bond provided that at least one of R₁, R₂, R₃ and R₄ is halogen.

2. A composition as claimed in claim 1 in which at least one of R₁, R₂, R₃ and R₄ is bromine.

3. A composition as claimed in claim 1 or claim 2 in which at least one of R₃ and R₄ is Br.

4. A composition as claimed in any one of claims 1 to 3 in which cell-permeabilising agent is selected from the group consisting of antibiotics, aldehydes, biguanides, halogen releasing agents, peroxygens, phenols, bis-phenols, quaternary ammonium compounds, alcohols, glycols, ionic and non-ionic detergents.

5. A composition as claimed in claim 4 in which cell-permeabilising agent is selected from the group consisting of Polymyxin B, Glutaraldehyde, Formaldehyde, Chlorhexidine, Hypochlorous acid, Iodine, Hydrogen peroxide, Peracetic acid, Chlorinated bis-phenol fenticlor, Hexachlorophene, Cetyltrimethylammonium bromide (CTAB), Tetrabutylammoniumhydrogen sulfate, Didecyldimethylammonium bromide, Cetylpyridium chloride, Toluene, Polyethylene glycol (PEG), Ethylenediaminetetraacetic acid (EDTA), Diamidines, Citric acid, Sodium lauryl sulfate (SDS), TritonX-100 and Tween 80

6. A composition as claimed in claim 5 in which cell-permeabilising agent is selected from the group consisting of Polymyxin B, EDTA, citric acid, tetrabutylammoniumhydrogen sulfate, didecyldimethylammonium bromide and Tween 80.

7. A composition as claimed in any one of claims 1 to 6 in which the compound is selected from the group consisting of 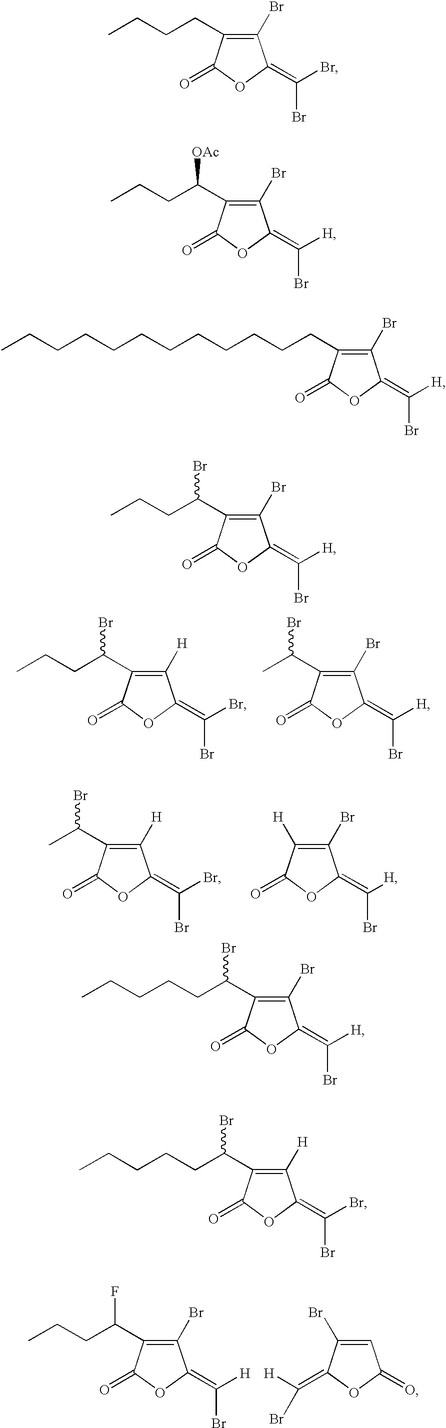 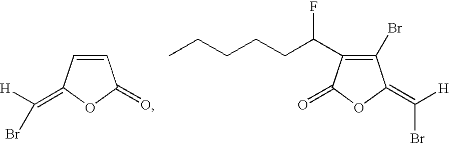
and combination thereof.

8. A method of manufacturing an antimicrobial composition, the method comprising combining a cell-permeabilising agent with and a pharmaceutically acceptable diluent with at least one compound of general formula I: 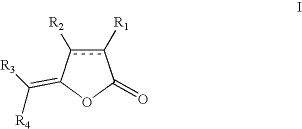
wherein R₁ and R₂ are independently H, halogen, alkyl, alkoxy, oxoalkyl, alkenyl, aryl or arylalkyl whether unsubstituted or substituted, optionally interrupted by one or more heteroatoms, straight chain or branched chain, hydrophilic or fluorophilic;
R₃ and R₄ are independently H, halogen, alkyl, aryl or arylalkyl, alkoxy;
R₃ or R₄+R₂ can be a saturated or an unsaturated cycloalkane;
and “ [symbol] ” represents a single bond or a double bond provided that at least one of R₁, R₂, R₃ and R₄ is halogen;

9. A method as claimed in claim 8 in which at least one of R₁, R₂, R₃ and R₄ is bromine.

10. A method as claimed in claim 8 or claim 9 in which at least one of R₃ and R₄ is Br.

11. A method as claimed in any one of claims 8 to 10 in which cell-permeabilising agent is selected from the group consisting of antibiotics, aldehydes, biguanides, halogen releasing agents, peroxygens, phenols, bis-phenols, quaternary ammonium compounds, alcohols, glycols, ionic and non-ionic detergents.

12. A method as claimed in claim 11 in which cell-permeabilising agent is selected from the group consisting of Polymyxin B, Glutaraldehyde, Formaldehyde, Chlorhexidine, Hypochlorous acid, Iodine, Hydrogen peroxide, Peracetic acid, Chlorinated bis-phenol fenticlor, Hexachlorophene, Cetyltrimethylammonium bromide (CTAB), Tetrabutylammoniumhydrogen sulfate, Didecyldimethylammonium bromide, Cetylpyridium chloride, Toluene, Polyethylene glycol (PEG), Ethylenediaminetetraacetic acid (EDTA), Diamidines, Citric acid, Sodium lauryl sulfate (SDS), TritonX-100 and Tween 80

13. A method as claimed in claim 12 in which cell-permeabilising agent is selected from the group consisting of Polymyxin B, EDTA, citric acid, tetrabutylammoniumhydrogen sulfate, didecyldimethylammonium bromide and Tween 80.

14. A method as claimed in any one of claims 8 to 13 in which the compound is selected from the group consisting of 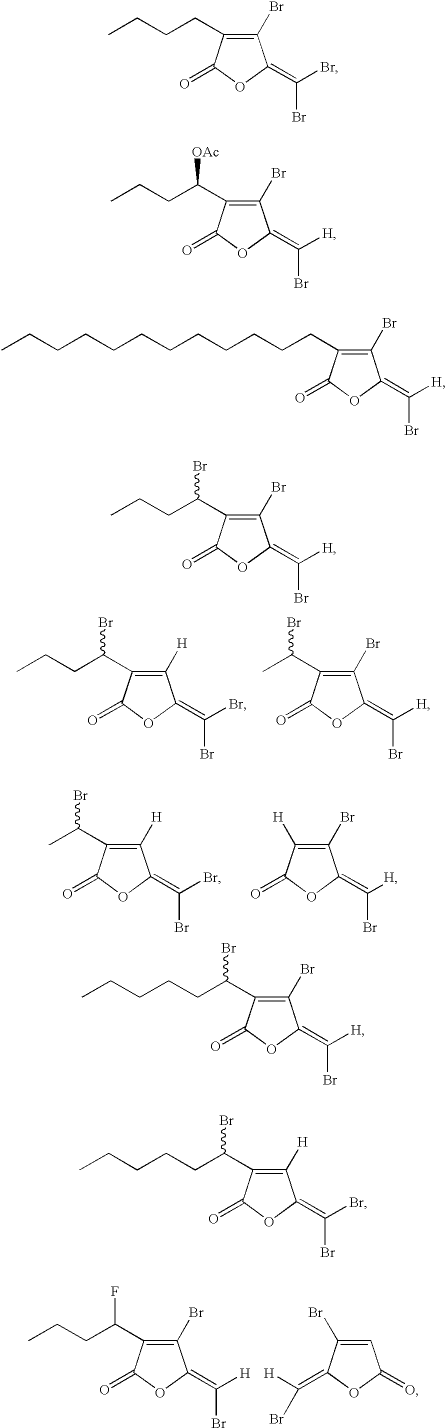 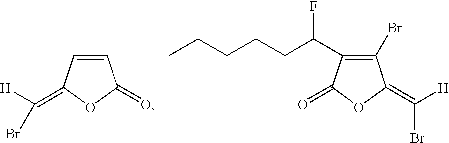
and combinations thereof.

15. A method of inhibiting the growth of a microorganism, the method comprising exposing the microorganism to an effective amount of an antimicrobial composition according to any one of claims 1 to 7 for sufficient time such that the microorganism is inhibited.

16. A method of treating microbial infection or decreasing the severity of symptoms of microbial infection in an animal, the method comprising administering to the animal an effective amount of the composition as claimed in any one of claims 1 to 7.

17. A method as claimed in claim 16 in which the microbial infection is Pseudomonas infection or Candida infections.

18. A method of treating Pseudomonas infection in an animal, the method comprising administering to an animal in need of such treatment a composition comprising tobramycin and at least one compound of general formula I: 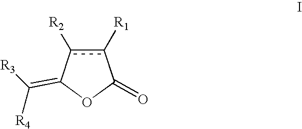
wherein R₁ and R₂ are independently H, halogen, alkyl, alkoxy, oxoalkyl, alkenyl, aryl or arylalkyl whether unsubstituted or substituted, optionally interrupted by one or more heteroatoms, straight chain or branched chain, hydrophilic or fluorophilic;
R₃ and R₄ are independently H, halogen, alkyl, aryl or arylalkyl, alkoxy;
R₃ or R₄+R₂ can be a saturated or an unsaturated cycloalkane;
and “ [symbol] ” represents a single bond or a double bond provided that at least one of R₁, R₂, R₃ and R₄ is halogen.

19. A method as claimed in claim 18 in which at least one of R₁, R₂, R₃ and R₄ is bromine.

20. A method as claimed in claim 18 or claim 19 in which at least one of R₃ and R₄ is Br.

21. A method as claimed in any one of claims 18 to 20 in which the compound is selected from the group consisting of 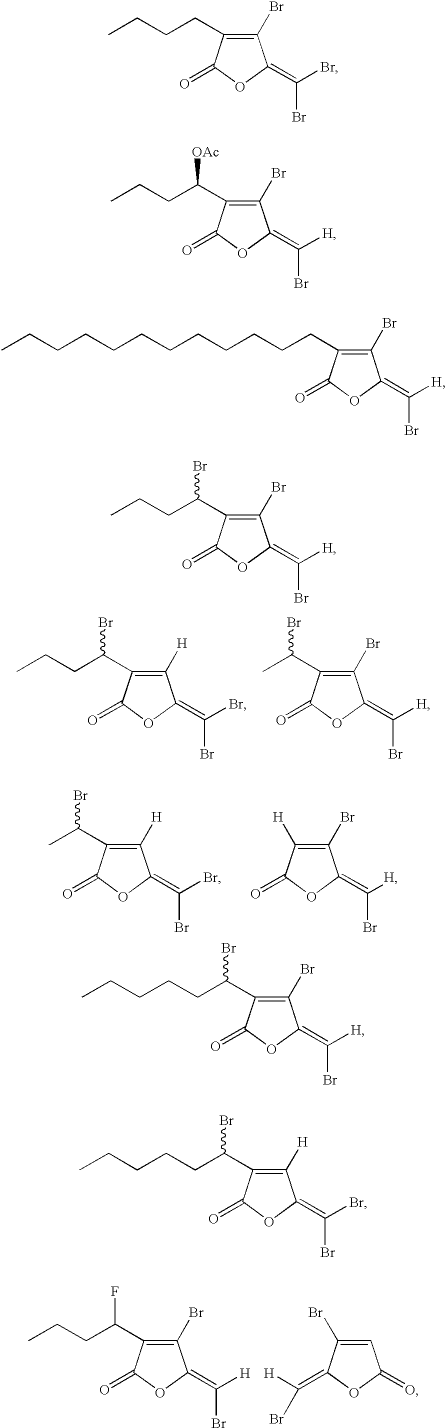 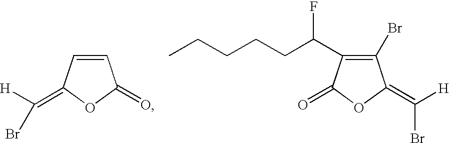
and combinations thereof.

22. A method as claimed in any one of claims 18 to 21 in which the Pseudomonas infection is P. aeruginosa infection.

23. A method as claimed in any one of claims 18 to 22 in which the Pseudomonas infection is a lung infection.

24. A method as claimed in any one of claims 18 to 22 in which the animal is human.

25. A method as claimed in claim 24 in which the animal is suffering from cystic fibrosis.

26. A composition for use in treatment of Pseudomonas infection, the composition comprising tobramycin and at least one compound of general formula I: 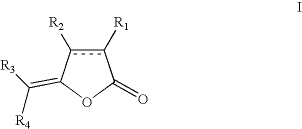
wherein R₁ and R₂ are independently H, halogen, alkyl, alkoxy, oxoalkyl, alkenyl, aryl or arylalkyl whether unsubstituted or substituted, optionally interrupted by one or more heteroatoms, straight chain or branched chain, hydrophilic or fluorophilic;
R₃ and R₄ are independently H, halogen, alkyl, aryl or arylalkyl, alkoxy;
R₃ or R₄+R₂ can be a saturated or an unsaturated cycloalkane;
and “ [symbol] ” represents a single bond or a double bond provided that at least one of R₁, R₂, R₃ and R₄ is halogen.

27. A composition as claimed in claim 26 in which at least one of R₁, R₂, R₃ and R₄ is bromine.

28. A composition as claimed in claim 26 or claim 27 in which at least one of R₃ and R₄ is Br.

29. A method as claimed in any one of claims 26 to 28 in which the compound is selected from the group consisting of 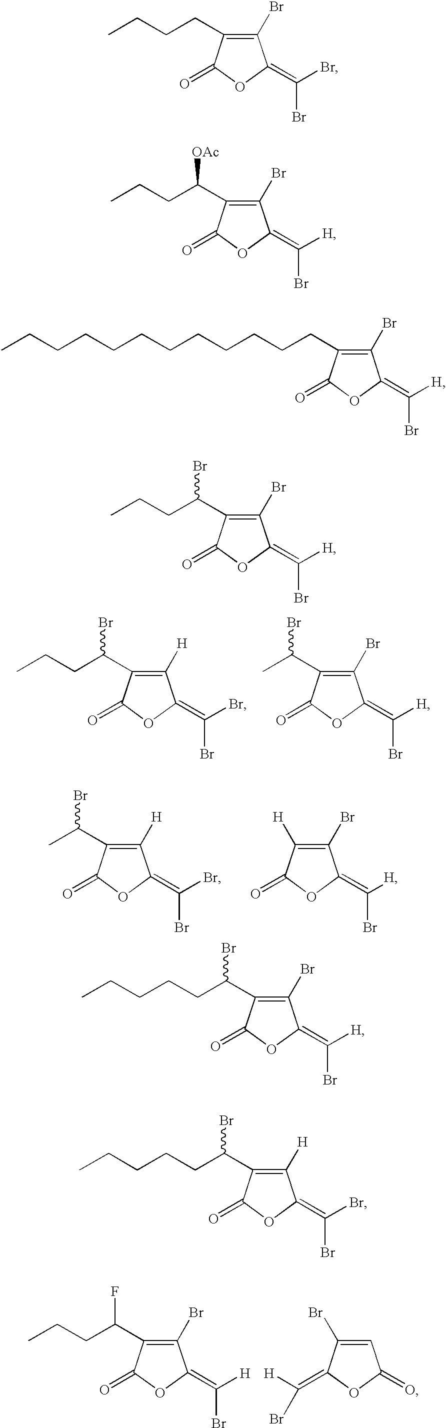 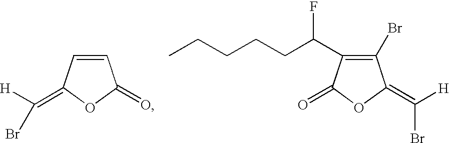
and combinations thereof.

30. A composition as claimed in any one of claims 28 to 29 in which the Pseudomonas infection is P. aeruginosa infection.

31. A composition as claimed in any one of claims 26 to 30 in which the Pseudomonas infection is a lung infection.

32. A composition as claimed in any one of claims 26 to 31 in which the animal is human.

33. A composition as claimed in claim 32 in which the animal is suffering from cystic fibrosis.

34. A contact lens cleaning preparation comprising the composition as claimed in any one of claims 1 to 7.

35. A washing solution comprising the composition as claimed in any one of claims 1 to 7.

36. A mouth wash preparation comprising the composition as claimed in any one of claims 1 to 7.

37. A disinfectant preparation comprising the composition as claimed in any one of claims 1 to 7.

38. A dentifrice comprising the composition as claimed in any one of claims 1 to 7.

39. An animal feedstock supplement comprising the composition as claimed in any one of claims 1 to 7.

40. A cleaning preparation comprising the composition as claimed in any one of claims 1 to 7.

41. A method of cleaning a surface which comprises applying to the surface the composition as claimed in any one of claims 1 to 7.

42. A method as claimed in claim 41 in which the surface to be cleaned is a hard surface, woven surface or non-woven surface.

43. A method as claimed in claim 41 or 42 in which the surface to be cleaned is a toilet bowl, bath tub, drain, countertop, food surface, airduct, air conditioner, carpet or cloth.

44. A topical dressing for burns comprising the composition as claimed in any one of claims 1 to 7.

45. A paint comprising the composition as claimed in any one of claims 1 to 7.

46. A skin cream preparation comprising the composition as claimed in any one of claims 1 to 7.
